(19) United States
(12) Patent Application Publication
(10) Pub. No.: US 2017/0303792 A1
(43) Pub. Date: Oct. 26, 2017

(54) OBJECT INFORMATION ACQUIRING APPARATUS AND OBJECT INFORMATION ACQUIRING METHOD

(71) Applicant: CANON KABUSHIKI KAISHA, Tokyo (JP)

(72) Inventor: Daisuke Nagao, Kawaguchi-shi (JP)

(21) Appl. No.: 15/488,625

(22) Filed: Apr. 17, 2017

(30) Foreign Application Priority Data
Apr. 26, 2016 (JP) 2016-088025

(51) Int. Cl. A61B 5/00; G01S 15/89; G01S 7/52

(52) U.S. Cl. CPC A61B 5/0095; G01S 15/8965; G01S 7/52085; A61B 5/4312; A61B 5/708; A61B 2576/00

(57) ABSTRACT

An object information acquiring apparatus is used which has an irradiating unit irradiating an object with light with a first wavelength at a first irradiation frequency, an element receiving an acoustic wave generated by the object irradiated with the light to output an electric signal, a processing unit acquiring characteristic information on the object using the electric signal, a scanning unit changing position of the irradiating unit relative to the object, and a controlling unit controlling movement of the scanning unit. The controlling unit controls the scanning unit such that an amount of light to which an identical area of the object is exposed is larger than a smallest value of a maximum permissible exposure at the first irradiation frequency.

### BACKGROUND OF THE INVENTION

### Field of the Invention

The present invention relates to an object information acquiring apparatus and an object information acquiring method.

### Description of the Related Art

In the medical field, active research is ongoing on optical imaging techniques in which an object such as a living organism is irradiated with light from a light source such as a laser, and information on the inside of the object obtained based on the light having entered the object is imaged. Photoacoustic imaging (PAI) is one such optical imaging technique. In photoacoustic imaging, an object is irradiated with pulsed light generated by a light source, an object tissue absorbs energy of the pulsed light propagating and diffusing through the object and generates an acoustic wave, the acoustic wave is received, and based on this signal corresponding to the received acoustic wave, object information is imaged.

PAI utilizes a difference in optical energy absorptance between a target segment such as a tumor and other tissues. Specifically, when a target segment absorbs optical energy radiated to the target segment and thus expands instantaneously, the target segment generates an elastic wave (photoacoustic wave), and a probe receives this elastic wave. Mathematical analysis of the received signal provides information on the inside of the object (particularly, an initial sound pressure distribution, an optical energy absorption density distribution, and an absorption coefficient distribution). These information can also be utilized for quantitative measurement of a particular substance in the object, for example, oxygen saturation in the blood. In recent years, considerable efforts have been poured into pre-clinical studies in which blood vessels in small animals are imaged using this photoacoustic imaging, as well as clinical studies in which the principle of the former is applied to diagnoses of breast cancer and the like (“Photoacoustic Tomography: In Vivo Imaging From Organelles to Organs”, Lihong V. Wang Song Hu, Science 335, 1458 (2012)).

Japanese Patent Application Laid-open No. 2012-179348 indicates that a probe is scanned in one plane, then moved in a direction perpendicular to the scanning plane to be scanned in another plane, and that such scanning is repeated a plurality of times. According to Japanese Patent Application Laid-open No. 2012-179348, such a scanning method is used to allow object information to be acquired at a high resolution over a wide range.
Patent Literature 1: Japanese Patent Application Laid-open No. 2012-179348
Non Patent Literature 1: “Photoacoustic Tomography: In Vivo Imaging From Organelles to Organs”, Lihong V. Wang Song Hu, Science 335, 1458 (2012)

### SUMMARY OF THE INVENTION

For apparatuses using the photoacoustic imaging technique, the signal-to-noise ratio (SNR) of received signals is desirably increased to enhance contrast. To achieve this, noise may be reduced by increasing the number of times that received signals are acquired for averaging. However, a simple increase in the number of times that received signals are acquired correspondingly extends the time for acquisition, leading to an increased time for which the subject is restrained.

To increase the number of times that signals are acquired while suppressing an increase in measurement time, an irradiation frequency of laser light may be increased (that is, the numbers of times that light is radiated and signals are acquired per unit time may be increased). However, Japanese Industrial Standards (JIS) C6802 or International Standards IEC 60825-1 specify a maximum permissible exposure (MPE). This may be considered to be the maximum permissible value, for each pulse, of exposure to pulsed light repeatedly radiated to an identical irradiation area on the skin. According to the standards, for light of 756 nm, the MPE is largest when the irradiation frequency is approximately 10 Hz or lower. If the irradiation frequency is higher than 10 Hz and is, for example, 20 Hz, then as depicted in FIG. 2, the MPE starts decreasing in inverse proportion to the exposure time at a time point where a certain exposure time (in FIG. 2, 3.8 seconds) elapses. Then, from a time point where another exposure time (in FIG. 2, 10 seconds) further elapses, the MPE becomes a constant value.

When the pulsed light has a constant irradiation area, an initial sound pressure p of a photoacoustic wave is expressed by Equation (1).

p=Γμaφ (1)

In the equation, Γ: Grueneisen constant, μa: absorption coefficient, and φ: the amount of light. Equation (1) indicates that as the amount of light φ radiated to the tissue (light absorber) inside the object decreases in inverse proportion to the exposure time, the initial sound pressure p of the photoacoustic wave also decreases in inverse proportion to the exposure time. FIG. 2 indicates that, when the irradiation frequency of the pulsed light radiated to the object is 20 Hz, if the exposure time is 3.8 seconds or more, irradiation density (the amount of light radiated per unit area) needs to be reduced from a time point where 3.8 seconds elapse. From a time point where 10 seconds elapse, the irradiation density needs to be reduced to approximately half of the irradiation density when the irradiation frequency is 10 Hz.

Hence, while an increased irradiation frequency is effective for reducing noise based on the averaging, it also reduces unfavorably a reception sound pressure obtained when discrete signals are acquired. Light having entered the object attenuates exponentially due to scattering and absorption. Thus, a reduced irradiation density leads to a significant reduction in the amount of light reaching a deep portion of the object. This may make the SNR increase effect difficult to produce.

The present invention has been developed in consideration of the above-described problem. An object of the present invention is to increase the SNR.

The present invention provides an object information acquiring apparatus comprising:

an irradiating unit that irradiates an object with light with a first wavelength at a first irradiation frequency;

an element that receives an acoustic wave generated by the object irradiated with the light to output an electric signal;

a processing unit that acquires characteristic information on the object using the electric signal;

a scanning unit that changes a position of the irradiating unit relative to the object; and

a controlling unit that controls the scanning unit,

wherein the controlling unit controls the scanning unit such that an amount of light to which an identical area of the object is exposed is larger than a smallest value of a maximum permissible exposure at the first irradiation frequency.

The present invention also provides an object information acquiring method comprising:

an irradiating step of irradiating, by an irradiating unit, an object with light with a first wavelength at a first irradiation frequency;

a receiving step of receiving, by an element, an acoustic wave generated by the object irradiated with the light to output an electric signal;

a processing step of acquiring characteristic information on the object using the electric signal;

a scanning step of varying, by a scanning unit, a position of the irradiating unit relative to the object; and

a controlling step of controlling the scanning unit,

wherein the controlling step controls the scanning unit such that an amount of light to which an identical area of the object is exposed is larger than a smallest value of a maximum permissible exposure at the first irradiation frequency.

The aspects of the present invention increase the SNR.

Further features of the present invention will become apparent from the following description of exemplary embodiments with reference to the attached drawings.

### BRIEF DESCRIPTION OF THE DRAWINGS

FIG. 1 is a diagram depicting a configuration of a photoacoustic apparatus according to the present invention;

FIG. 2 is a diagram illustrating an MPE;

FIG. 3 is a diagram illustrating an example of a reception directionality of an acoustic-wave receiving element 300 according to the present invention;

FIG. 4 is a diagram illustrating operations of the photoacoustic apparatus according to Embodiment 1 of the present invention;

FIGS. 5A and 5B are diagrams illustrating a scanning trajectory according to Embodiment 1 of the present invention;

FIGS. 6A and 6B are diagrams illustrating a scanning trajectory according to Embodiment 2 of the present invention;

FIG. 7 is a diagram illustrating operations of the photoacoustic apparatus according to Embodiment 3 of the present invention;

FIG. 8 is a diagram illustrating a scanning trajectory according to Embodiment 3 of the present invention;

FIGS. 9A to 9C are diagrams illustrating a part of the scanning trajectory according to Embodiment 3 of the present invention in detail; and

FIG. 10 is a diagram illustrating a part of the scanning trajectory according to Embodiment 3 of the present invention in detail.

### DESCRIPTION OF THE EMBODIMENTS

Preferred embodiments of the present invention will be described below with reference to the drawings. The dimensions, materials, shapes, relative arrangements, and the like of components described below should be varied as needed in accordance with a configuration of an apparatus to which the present invention is applied and various conditions for the apparatus. Therefore, the scope of the present invention is not intended to be limited to the following description.

The present invention relates to a technique of detecting an acoustic wave propagating from an object to generate and acquire characteristic information on the inside of the object. Therefore, the present invention is considered to be an object information acquiring apparatus or a control method therefor or an object information acquiring method or a signal processing method. The present invention is also considered to be a program that allows an information processing apparatus equipped with a hardware resource such as a CPU and a memory to execute the above-described methods, or a storage medium that stores this program.

The object information acquiring apparatus in the present invention includes an apparatus that irradiates an object with light (electromagnetic wave) to cause an acoustic wave to be generated inside the object and that receives the acoustic wave to acquire characteristic information on the object in the form of image data. In this case, the characteristic information is information that is generated using received signals resulting from reception of the photoacoustic waves and that relates to characteristic values corresponding to the respective positions in the object.

Characteristic information derived from an electric signal (photoacoustic signal) resulting from photoacoustic measurement is a value representing the absorptance of the optical energy. The characteristic information includes information relating to, for example, a source of acoustic waves resulting from light irradiation, the initial sound pressure inside the object, or the absorption density and coefficient of optical energy derived from the initial sound pressure, and the concentrations of substances forming the tissue.

For the substance concentrations, an oxygenated hemoglobin concentration and a reduced hemoglobin concentration are determined to allow calculation of information on an oxygen saturation distribution. A glucose concentration, a collagen concentration, a melanin concentration, volume fractions of fat and water, and the like are also determined.

Based on characteristic information for each position in the object, a two- or three-dimensional characteristic information distribution is obtained. Distribution data may be generated as image data. Characteristic information may be determined in the form of distribution information for each position in the object rather than in the form of numerical data. Examples of the distribution information include an initial sound pressure distribution, an energy absorption density distribution, an absorption coefficient distribution, and an oxygen saturation distribution.

The acoustic wave as used herein is typically an ultrasonic wave and includes an elastic wave referred to as a sound wave or an acoustic wave. An electric signal into which an acoustic wave is converted by a probe or the like is referred to as an acoustic signal. The term “ultrasonic wave” or “acoustic wave” as used herein is not intended to limit the wavelengths of these elastic waves. An acoustic wave resulting from a photoacoustic effect is referred to as a photoacoustic wave. An electric signal derived from the photoacoustic wave is referred to as a photoacoustic signal.

### Embodiment 1

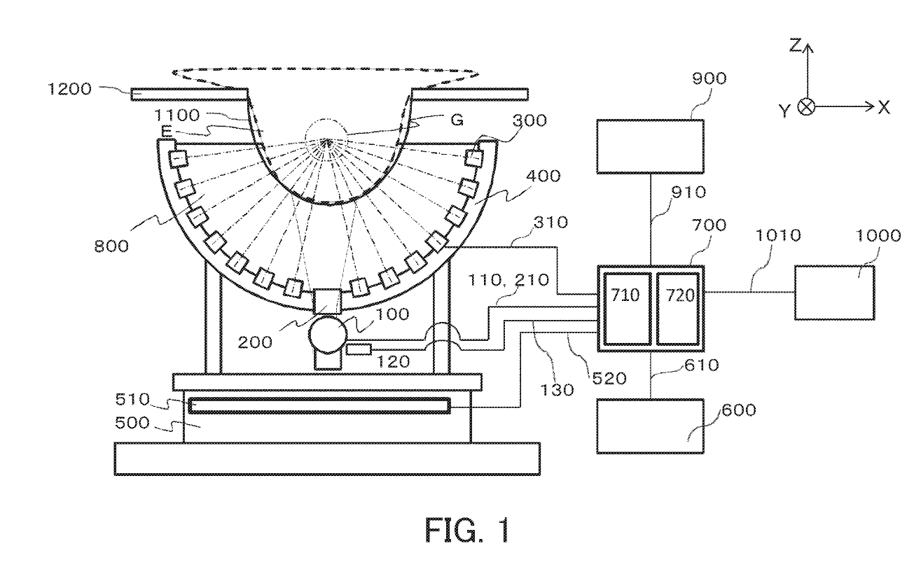

FIG. 1 is a schematic diagram of a photoacoustic apparatus that is an object information acquiring apparatus according to the present embodiment. The apparatus acquires information (object information) such as optical characteristics of an object E based on a received signal (photoacoustic signal) of a photoacoustic wave resulting from the photoacoustic effect.

<Basic Configuration>

The photoacoustic apparatus in the present embodiment includes a light source 100, a light irradiation detector 120, an optical system 200, a plurality of acoustic wave-receiving elements 300, a supporting body 400, a scanner 500 serving as a scanning unit, and a scanning position sensor 510. The photoacoustic apparatus in the present embodiment further includes a shape acquiring unit 600, a computer 700, a display 900 serving as a display unit, an input unit 1000, a shape holding unit 1100, and a mounting unit 1200. Components of the photoacoustic apparatus and components used for measurement will be described below.

(Object)

The object E is a measurement target. Specific examples of the object E include a living organism such as the breast and a phantom used to adjust the apparatus and simulating the acoustic and optical characteristics of the living organism. Specific examples of the acoustic characteristics include the propagation speed and attenuation rate of the acoustic wave. Specific examples of the optical characteristics include the absorption and scattering coefficients of light. A light absorber with a large optical absorption coefficient is present inside the object. For living organisms, examples of the light absorber include hemoglobin, water, melanin, collagen, and lipid. The phantom has a substance with simulated optical characteristics sealed therein as a light absorber. In FIG. 1, the object E is depicted by a dashed line.

(Light Source)

The light source 100 is an apparatus that generates pulsed light. The light source is desirably a laser in order to obtain high power. The light source may be a flash lamp, a light emitting diode, or a laser diode. To allow a photoacoustic wave to be effectively generated, the object is preferably irradiated with light in a sufficiently short time in accordance with thermal characteristics of the object. When the object is a living organism, pulsed light generated by the light source 100 desirably has a pulse width of not more than several tens of nanoseconds. The pulsed light desirably has a near infrared region (for example, approximately 700 nm to 1200 nm) referred to as a biological optical window. Light in this region can reach a relatively deep portion of the living organism, allowing information on the deep portion to be obtained. When the measurement is limited to a surface portion of the living organism, light within the range from a visible region of approximately 500 to 700 nm to a near infrared region may be used. Moreover, the pulsed light desirably has a wavelength corresponding to a high absorption coefficient for an observation target. A timing when the light source 100 generates pulsed light is controlled by the computer 700 via a control line 110.

(Optical System)

The optical system 200 is an apparatus that guides pulsed light generated by the light source 100 to the object E. For example, optical equipment such as a lens, a mirror, a prism, an optical fiber, or a diffuser may be used. When light is guided, any of these types of optical equipment may be used to vary the shape or optical density of light so as to provide a desired optical distribution. The optical system 200 in the present embodiment is configured to illuminate the area of the center of curvature of the hemispherical supporting body 400 having a hemispherical inner surface. The optical system corresponds to an irradiating unit in the present invention.

(Light Irradiation Detector)

The light irradiation detector 120 is an apparatus that detects emission of light from the light source 100. A part of the optical fiber (not depicted in the drawings) present in the optical system 200 is branched such that a photodiode mounted in the light irradiation detector 120 detects light emission. A detection signal from the light irradiation detector 120 is transmitted to the computer 700 via the control line 210 as a light emission timing. A method for detecting the light emission timing is not limited to this. Detecting means other than the photodiode may be utilized. For example, the light emission timings may be acquired using a clock signal or an output from a counter circuit that controls the light emission from the light source 100.

(Acoustic-Wave Receiving Element)

The acoustic-wave receiving element 300 receives and converts a photoacoustic wave into an electric signal. The acoustic-wave receiving element 300 desirably has a high receiving sensitivity to photoacoustic waves from the object E and has a wide frequency band. A member forming the acoustic-wave receiving element 300 may be a piezoelectric ceramic material represented by lead zirconate titanate (PZT) or a piezoelectric polymer film material represented by polyvinylydene fluoride (PVDF). Alternatively, any element other than the piezoelectric element may be used. For example, a capacitive element such as a capacitive micro-machined ultrasonic transducer (CMUT) or an acoustic-wave receiving element including a Fabry-Perot interferometer may be utilized.

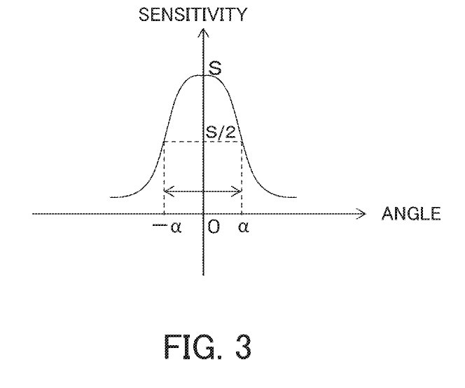

FIG. 3 illustrates an example of receiving sensitivity characteristics of the acoustic-wave receiving element 300. The receiving sensitivity characteristics illustrated in FIG. 3 represent a receiving directionality determined by an incident angle between a normal direction of a receiving surface of the acoustic-wave receiving element 300 and an incident direction of a photoacoustic wave. In the example in FIG. 3, the receiving density is highest for a photoacoustic wave incident in the normal direction of the receiving surface and decreases with increasing incident angle. The acoustic-wave receiving element 300 according to the present embodiment has a circular planar receiving surface. When the maximum value of the receiving sensitivity is denoted by S, the incident angle is denoted by α when the receiving sensitivity is half the maximum value, i.e. S/2. In the present embodiment, a receiving area for sensitive reception corresponds to an area where a photoacoustic wave is incident on the receiving surface of the acoustic-wave receiving element 300 at an angle that is not more than the incident angle α. In FIG. 1, a direction in which the acoustic-wave receiving element 300 exhibits the highest receiving sensitivity is depicted by a long dashed short dashed line.

(Supporting Body)

The supporting body 400 is a generally hemispherical container. A plurality of the acoustic-wave receiving elements 300 is installed along an inner surface of the hemisphere, with the optical system 200 installed at a bottom portion (pole) of the hemisphere. The supporting body 400 in the present embodiment serves as a hemispherical container. An acoustic matching material 800 described below can be held on the inner side of the hemisphere. For example, the acoustic matching material 800 may be like a gel or may be water. The supporting body 400 is preferably formed of a metallic material with a high mechanical strength sufficient to support the above-described members.

The acoustic-wave receiving elements 300 provided on the supporting body 400 are arranged on the hemispherical surface such that the direction in which each of the elements exhibits the highest receiving sensitivity extends toward the center of curvature of the hemisphere. FIG. 1 is a sectional view, in an X-Z plane, of the hemispherical supporting body 400 taken along a central axis thereof. Long dashed short dashed lines converging at a partial area in the object E indicate receiving directions of the acoustic-wave receiving elements 300. This arrangement allows each of the acoustic-wave receiving elements 300 to sensitively receive a photoacoustic wave generated in a particular area. This particular area is hereinafter referred to as a “high sensitivity area”.

Object information obtained from received signals from the thus arranged acoustic-wave receiving elements 300 has a resolution that is highest at the center of curvature of the hemisphere and that decreases with increasing distance from the center of curvature. In the present embodiment, the high sensitivity area refers to an area from a point with the highest resolution to a point with a resolution that is half of the highest resolution. An area G enclosed by a long dashed double-short dashed line in FIG. 1 corresponds to the high sensitivity area.

The directions in which the acoustic-wave receiving elements exhibit the highest sensitivity need not intersect one another so long as the desired high sensitivity area can be formed. Furthermore, the directions in which at least some of the acoustic-wave receiving elements 300 supported by the supporting body 400 exhibit the highest receiving sensitivity need to extend toward a particular area so as to enable sensitive reception of a photoacoustic wave generated in the particular area. That is, the acoustic-wave receiving elements 300 need to be arranged on the supporting body 400 such that at least some of the acoustic-wave receiving elements 300 can sensitively receive a photoacoustic wave generated in the high sensitivity area. In other words, the acoustic-wave receiving elements 300 need to be arranged on the supporting body 400 such that the length of the directional axis of each of the acoustic-wave receiving elements 300 indicating the direction corresponding to the highest sensitivity is shorter than the distance between the acoustic-wave receiving elements 300. The shape of the supporting body 400 may be, instead of the hemisphere, a cup-like shape, a bowl-like shape, a part of an ellipsoid, a combination of curves or planes, or the like.

(Scanner)

The scanner 500 is an apparatus that varies the position of the supporting body 400 relative to the object E by moving the position of the supporting body 400 in an X direction and a Y direction in FIG. 1. Varying the position of the supporting body 400 relative to the object E varies the position of the irradiation area in the object E irradiated with light radiated to the object via the optical system 200. The scanner 500 includes a guide mechanism for the X and Y direction not depicted in the drawings and a driving mechanism for the X and Y directions. A scanning position sensor 510 described below receives the position of the supporting body 400 in the X and Y directions. As depicted in FIG. 1, since the supporting body 400 is loaded on the scanner 500, the guide mechanism is preferably a linear guide or the like, which can withstand a heavy load. The driving mechanism may be a lead screw mechanism, a link mechanism, a gear mechanism, a hydraulic mechanism, or the like. A motor or the like may be used to exert a driving force. The scanner corresponds to the scanning unit in the present invention.

In the present invention, the relative position between the object E and the supporting body 400 needs to be varied. Therefore, with the supporting body 400 fixed, the object E may be moved. When the object E is moved, a supporting unit (not depicted in the drawings) that supports the object E or the mounting unit 1200 may be moved to move the object E. Moreover, both the object E and the supporting body 400 may be moved. In the present embodiment, the driving mechanism is based on the two axes, that is, the X and Y axes. However, the driving mechanism may move in the three directions, that is, the X and Y directions and a Z direction. Any configuration may be used so long as at least one of the object E and the supporting body 400 is configured to be movable.

(Scanning Position Sensor)

The scanning position sensor 510 is means for acquiring position coordinate information on the supporting body 400 when the scanner 500 changes the position of the supporting body 400 relative to the object E. The scanning position sensor 510 acquires one-, two-, or three-dimensional position coordinate information on the supporting body 400 according to the configuration of the photoacoustic apparatus. The scanning position sensor 510 may be a potentiometer including a linear scale, a magnetic sensor, an infrared sensor, an ultrasonic sensor, an encoder, or a variable resistor. Any type of sensor may be used so long as the sensor can acquire one-, two-, or three-dimensional position coordinate information on the supporting body 400.

(Shape Acquiring Unit)

The shape acquiring unit 600 is an apparatus that acquires shape information representing the external shape of the object E or the shape holding unit 1100. The shape acquiring unit 600 may include an imaging apparatus that images the object E, such as a camera or a transducer array that transmits and receives acoustic waves. The transducer may be provided separately from the plurality of acoustic-wave receiving elements 300 or may be at least one of the plurality of acoustic-wave receiving elements 300. Such a transducer transmits an acoustic wave to receive the acoustic wave reflected by the object to obtain a received signal. Based on the received signal, a calculating unit 710 serving as a picked-up image processing unit acquires a picked-up image. Processing the picked-up image allows shape information on the object E to be acquired. The calculating unit 710 may acquire shape information on the object E based on images picked up in a plurality of directions using a three-dimensional measurement technique such as a stereo method. In this case, the imaging apparatus and the picked-up image processing unit are collectively referred to as the shape acquiring unit 600.

When the shape holding unit 1100 is used, the shape of the shape holding unit 1100 may be used as the shape information on the object E. The shape information on the shape holding unit 1100 may be stored in a storage unit 720 so that the shape acquiring unit 600 can read the information from the storage unit 720. The calculating unit 710 may also serve as the shape acquiring unit 600. If a plurality of the shape holding units is used, the shape information on each of the shape holding units is preferably stored in the storage unit 720. The shape holding unit to be used may be identified by the photoacoustic apparatus or specified by the user via the input unit 1000 so that the shape acquiring unit 600 can read from the storage unit 720 the shape information on the shape holding unit to be used. That is, the shape acquiring unit 600 selects one of a plurality of pieces of information on the respective shape holding units to acquire the selected shape information as shape information on the object. The shape acquiring unit 600 may be provided separately from the photoacoustic apparatus.

(Computer)

The computer 700 has the calculating unit 710 and the storage unit 720. The calculating unit 710 typically includes an element such as a CPU, a GPU, or an A/D converter or a circuit such as an FPGA or an ASIC. The calculating unit 710 may include an amplifying circuit for electric signals (received signals). The calculating unit may include a plurality of elements or circuits instead of a single element or circuit. Processing performed by the computer 700 may be executed by any of the elements or circuits. The storage unit 720 typically includes a storage medium such as a ROM, a RAM, or a hard disk. The storage unit may include a plurality of storage media instead of a single storage medium. The computer implements functions of a control unit and a processing unit in the present invention.

The calculating unit 710 executes signal processing on a plurality of electric signals output from the respective acoustic-wave receiving elements 300. Specifically, signal processing based on an image reconstruction algorithm is executed to allow characteristic information on the inside of the object to be acquired. In this case, it is possible to utilize the shape information on the object E acquired by the shape acquiring unit 600 or information on the scanning position corresponding to the timing of reception of a photoacoustic signal and detected by the scanning position sensor 510. The calculating unit 710 serving as the control unit can control operation of components of the photoacoustic apparatus via a bus. As described below, the calculating unit 710 serving as the control unit calculates a scanning trajectory and a scanning speed corresponding to an input imaging area. The calculating unit 710 controls the timing when the light source 100 generates pulsed light. A control signal for the light source 100 provided by the calculating unit 710 is transmitted to the light source 100 via the bus and the control line 110 to control the pulsed light generation timing for the light source 100. The calculating unit serving as the control unit controls movement of the scanner serving as the scanning unit.

The calculating unit 710 acquires position coordinate information on the supporting body 400 from the scanning position sensor 510. An information signal for the supporting body 400 provided by the calculating unit 710 is transmitted via the bus and a signal line 520 to allow the calculating unit to acquire position coordinate information.

The computer 700 is preferably configured to allow a plurality of signals to be simultaneously processed in a pipelined manner. This enables a reduction in the amount of time until object information is acquired. Different processes executed by the computer 700 may be saved to the storage unit 720 as respective programs to be performed by the calculating unit 710. However, the storage unit 720 to which the programs are saved is a non-transitory recording medium.

(Display)

The display 900 is an apparatus that displays object information output from the computer 700 in the form of distribution images, numerical data on a particular area of interest, and the like. The display 900 is typically a liquid crystal display but may be of another type such as a plasma display, an organic EL display, or an FED. The display 900 may be provided separately from the photoacoustic apparatus in the present embodiment.

(Input Unit)

The input unit 1000 is a member configured to accept inputs of desired information or specifications provided to the computer 700 by the user. The input unit 1000 may be a keyboard, a mouse, a touch panel, dials, buttons, and the like. When a touch panel is adopted as the input unit 1000, the display 900 may also serve as the input unit 1000.

(Shape Holding Unit)

The shape holding unit 1100 is a member that keeps the shape of the object E constant. The shape holding unit 1100 is attached to the mounting unit 1200. A plurality of the shape holding units may be replaced with one another according to the shape of the object E. When the object E is irradiated with light via the shape holding unit 1100, the shape holding unit 1100 is preferably transparent to the irradiation light. A material for the shape holding unit 1100 may be, for example, polymethylpentene or polyethylene terephthalate. Alternatively, the shape holding unit 1100 may be formed of a film or a net.

When the object E is the breast, the shape holding unit 1100 may be a member having a shape resulting from cutting of a sphere along a certain section or a cup-shaped member in order to suppress deformation of the breast shape to keep the shape constant. The shape of the shape holding unit 1100 may be designed as needed according to the volume of the object or the desired shape of the held object. For example, the shape holding unit 1100 may fit the external shape of the object E and may be substantially similar in shape to the object E.

(MPE)

The intensity of light permitted to be radiated to the biological tissue is specified as the maximum permissible exposure (MPE). Examples of safety standards include “IEC 60825-1: Safety of laser products”, “JIS C 6802: Safety Standards for Laser Products”, “FDA: 21CFR Part 1040.10, ANSI 2136.1: Laser Safety Standards”.

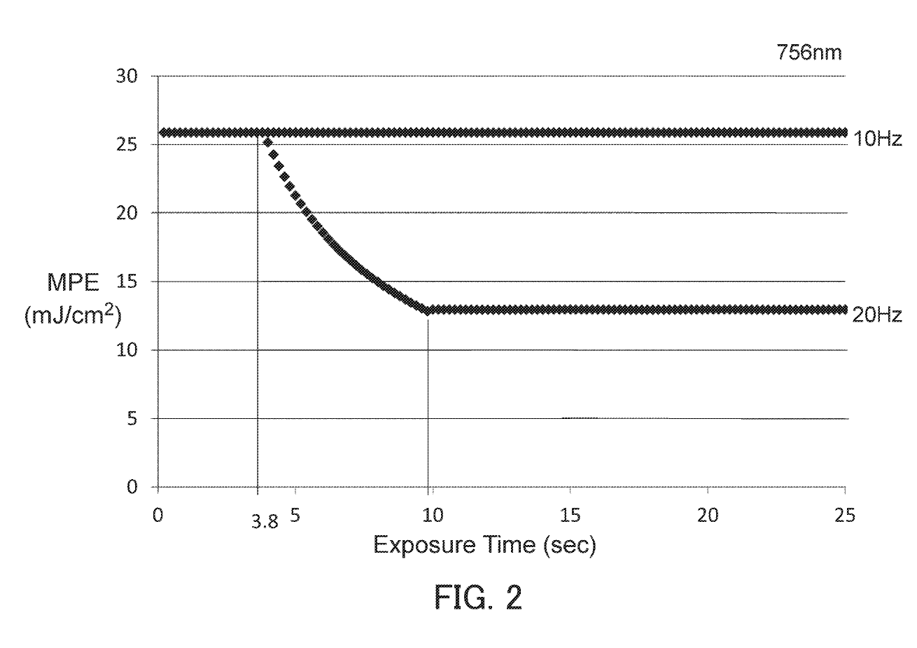

The maximum permissible exposure specifies the intensity of light permitted to be radiated per unit area. FIG. 2 indicates the MPE at a wavelength of 756 nm. According to the specification, the MPE is largest when the irradiation frequency (the number of irradiations per unit time) is approximately 10 Hz or lower. At a higher irradiation frequency, when an identical irradiation area is irradiated with repeated pulsed light, the exposure for each pulse needs to start to be reduced at a certain timing in inverse proportion to time. When the irradiation frequency at which the object is irradiated with pulsed light is changed from 10 Hz to 20 Hz, the irradiation density for each pulse (the amount of light radiated per unit time) needs to start to be attenuated from a time point where 3.8 seconds elapse and to be reduced, from a time point where 10 seconds elapse, to half of the irradiation density at the start of irradiation.

At a wavelength of 756 nm, the maximum irradiation time is 3.8 seconds for which light with the maximum irradiation density permitted for an identical area can be radiated at 20 Hz. Therefore, in the specification, for an irradiation frequency of 20 Hz, the highest MPE is defined to be the MPE obtained before 3.8 seconds have elapsed, and the lowest MPE is defined to be the MPE obtained at or after a time point where 10 seconds elapse. In the specification, a wavelength of 756 nm is designated as a first wavelength, a wavelength of 20 Hz is designated as a first irradiation frequency, and a wavelength of 10 Hz is designated as a second irradiation frequency. In this case, the lowest MPE may be referred to as the smallest value of the maximum permissible exposure at the first irradiation frequency. In this case, a first period refers to a period in which the maximum permissible exposure at the first irradiation frequency is higher than the smallest value of the maximum permissible exposure at the first irradiation frequency. In the case in FIG. 2, the first period is 10 seconds. In this example, the first period needs to be not more than 10 seconds. More preferably, setting the first period to not more than 3.8 seconds ensures that the exposure is prevented from exceeding the MPE.

<Operations of the Photoacoustic Apparatus>

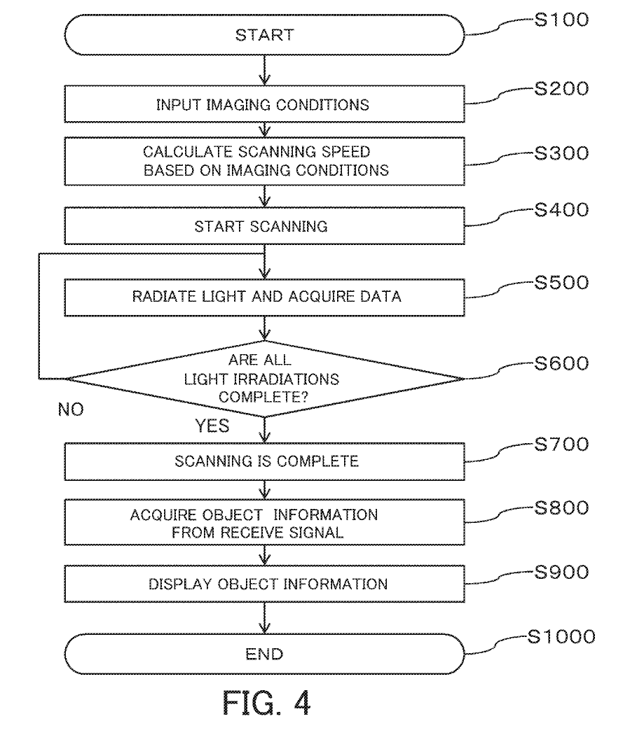

Now, using a flowchart in FIG. 4, operations of the embodiment of the present invention will be described.

(S100: Step of Starting Imaging)

The object E is inserted into the shape holding unit 1100. The space between the supporting body 400 and the shape holding unit 1100 and the space between the shape holding unit 1100 and the object E are filled with the acoustic matching material 800. Subsequently, the shape acquiring unit 600 acquires the shape information on the object E using the above-described method.

(S200: Step of Inputting Imaging Conditions)

Then, the user inputs imaging conditions. The input imaging conditions include an imaging area (area of interest), the irradiation frequency of pulsed light, the absorption coefficient, the scattering coefficient, a period in which photoacoustic waves generated inside the object E are acquired (hereinafter referred to as a “reception period”) or a timing for the acquisition, and desired image quality. Any other parameters needed to carry out imaging can be accepted. For the frequency and wavelength of pulsed light, selection instructions may be accepted via pull-down lists, radio buttons, or the like. If no area of interest is designated, an imaging area set by default may be used as a measurement area.

(S300: Step of Calculating the Scanning Speed Based on the Imaging Conditions)

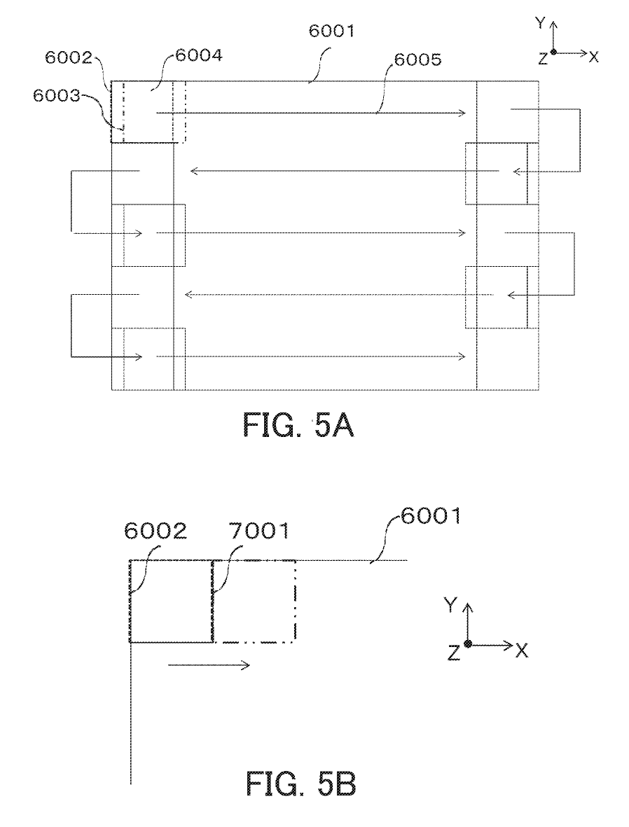

Now, a step of calculating the scanning speed based on the input imaging conditions will be described. FIG. 5A illustrates a scanning trajectory along which the input imaging area is scanned. FIG. 5B is an excerpt of a part of the scanning trajectory, and illustrates two irradiation areas irradiated with the respective pulsed light beams and completely separated from each other.

An imaging area 6001 is irradiated with pulsed light so as to allow a photoacoustic wave to be received for photoacoustic measurement. An area 6002 depicted by a dashed line is an irradiation area irradiated with a first pulsed light beam. The irradiation area in the present embodiment is a 100 mm×100 mm square. An area 6003 depicted by a long dashed short dashed line is an irradiation area irradiated with the next pulsed light beam. In an area 6004, the areas 6002 and 6003 corresponding to the consecutive pulsed light beams overlap. Along a scanning trajectory 6005, the imaging area is scanned. An area 7001 depicted by a long dashed double-short dashed line in FIG. 5B is obtained when, during scanning, the irradiation area is completely separated from the irradiation area 6002. The scanning trajectory is not limited to the one in the present embodiment. The present embodiment may adopt either of a continuous movement scheme in which the supporting body 400 is continuously moved and a step and repeat scheme in which movement of the supporting body 400 is stopped each time pulsed light is radiated and a photoacoustic wave is received.

As seen in FIG. 2, when laser light has a wavelength of 756 nm and an irradiation frequency of 20 Hz, an identical area can be irradiated using a laser intensity equivalent to the laser intensity in the case of an irradiation frequency of 10 Hz, for 3.8 seconds since the start of irradiation. Therefore, if the irradiation area successfully moves to the irradiation area 7001, which does not overlap the irradiation area 6002, before 3.8 seconds have elapsed since the irradiation of the irradiation area 6002, a laser light intensity equivalent to the intensity in the case of an irradiation frequency of 10 Hz can be achieved even at an irradiation frequency of 20 Hz. Therefore, the control unit sets the scanning speed to a value meeting this condition. Byway of example, the control unit may control the scanning unit so as to prevent overlapping of the irradiation areas irradiated with the light during two consecutive irradiations.

In the present embodiment, the irradiation area is 100 mm×100 mm in size. Since the irradiation frequency of laser light is 20 Hz, laser light can be radiated at an intensity corresponding to 10 Hz for 3.8 seconds. Therefore, the scanning speed needs to be higher than 100 mm/3.8 seconds. In this case, the irradiation area may be shaped like, instead of a square, an ellipse, a rectangle, or the like in association with the optical system. Furthermore, in order to efficiently generate photoacoustic waves, the intensity of laser light is preferably equal to the highest MPE. However, the effects of the present invention are produced so long as the intensity is higher than the lowest MPE.

As described above, determination of the scanning speed allows irradiation with light having an intensity higher than the smallest value of the MPE determined by the wavelength and irradiation frequency of pulsed light. As a result of increasing the intensity of irradiation light above the smallest value of the MPE utilizing movement of the irradiation area, the intensity (initial sound pressure) of photoacoustic waves is also increased, allowing generation of characteristic information images with excellent contrast. For example, laser light with a wavelength of 756 nm can be radiated using the highest MPE corresponding to 10 Hz even when the irradiation frequency of the laser light is set to be more than 10 Hz. Thus, the SNR can be improved.

With reference back to the flowchart, description will be continued.

(S400: Step of Starting Scanning)

After the scanning speed is calculated at S300, scanning is started. The user may give an instruction to start scanning or the computer 700 may automatically start scanning in response to calculation of the scanning speed.

(S500: Step of Radiating Light and Acquiring Data)

When scanning is started, the computer 700 outputs a control signal so as to allow the light source 100 to generate light at the desired timing in accordance with the irradiation frequency input in S200. The light is guided by the optical system 200 and radiated to the object E via the acoustic matching material 800. The light radiated to the object E is absorbed inside the object E to generate a photoacoustic wave. A data acquiring unit present inside the computer 700 and not depicted in the drawings acquires photoacoustic wave data during the reception period designated by the user in S200 and also acquires position coordinate information from the scanning position sensor 510.

(S600: Step of Determining whether or not Scanning and Light Irradiation are Complete)

The scanner 500 scans the imaging area set in S200 at the scanning speed calculated in S300. Then, S500 is repeated until scanning of all of the imaging area is complete. In step S600, the control unit determines whether or not photoacoustic measurement is complete for all of the imaging area based on the results of S500 immediately before S600. Upon determining that the photoacoustic measurement is not complete, the control unit returns to S500. Upon determining that the photoacoustic measurement is complete, the control unit advances to S700.

(S700: Step of Completing Scanning)

When the data acquisition under the imaging conditions set in S200 by the user is complete, the scanning is complete.

(S800: Step of Acquiring Object Information Based on Received Signals)

The calculating unit 710 serving as an information acquiring unit executes processing based on the image reconstruction algorithm on the digital signals acquired in S500 to acquire object information indicative of the characteristics of the object. The image reconstruction algorithm may be, for example, back projection in a time domain or a Fourier domain commonly used in conjunction with a tomography technique. If much time can be used for the reconstruction, an image reconstruction technique such as inverse problem analysis based on iterative processing can be utilized. So long as the desired image reconstruction is implemented, the image reconstruction algorithm is not limited to a particular algorithm.

(900: Step of Displaying the Object Information)

The display 900 displays the object information acquired in S800.

(S1000: Step of Ending the Imaging)

The imaging is ended.

As described above, determination of the scanning speed as in the case of Embodiment 1 enables irradiation with light having an intensity higher than the smallest value of the MPE determined by the wavelength and irradiation frequency of pulsed light.

For example, if light with a wavelength of 765 nm is radiated at an irradiation frequency of 20 Hz, when an identical irradiation area is irradiated with the light for 3.8 seconds or more, the intensity of the light radiated needs to be lower than the highest MPE (approximately 26 mJ/cm²). When the identical irradiation area is irradiated with the light for 10 seconds or more, the output needs to be reduced to the lowest MPE (approximately 13 mJ/cm²). However, utilization of movement of the irradiation area as in the case with the present embodiment, an output exceeding the lowest MPE can be output even at an irradiation frequency of 20 Hz. Thus, the SNR can be increased. As a result, the contrast of the resultant images is improved to enhance the capability of making diagnoses based on photoacoustic images.

In the present embodiment and other embodiments, the principle of the present invention can be applied to various wavelengths and irradiation frequencies of light. So long as light can be radiated at an intensity higher than the lowest MPE, the SNR increase effect of the present invention can be produced without irradiation at the highest MPE.

### Embodiment 2

In the description of Embodiment 1, the scanning trajectory is based on raster scanning in which main scanning and sub-scanning are repeated. In the description of Embodiment 2, the scanning trajectory is spiral. An apparatus configuration in Embodiment 2 is similar to the apparatus configuration in Embodiment and will thus not be described. A sequence of operations in Embodiment 2 is similar to the sequence of operations in Embodiment 1 except for S300 and will thus not be described.

(S300: Step of Calculating the Scanning Speed Based on the Imaging Conditions)

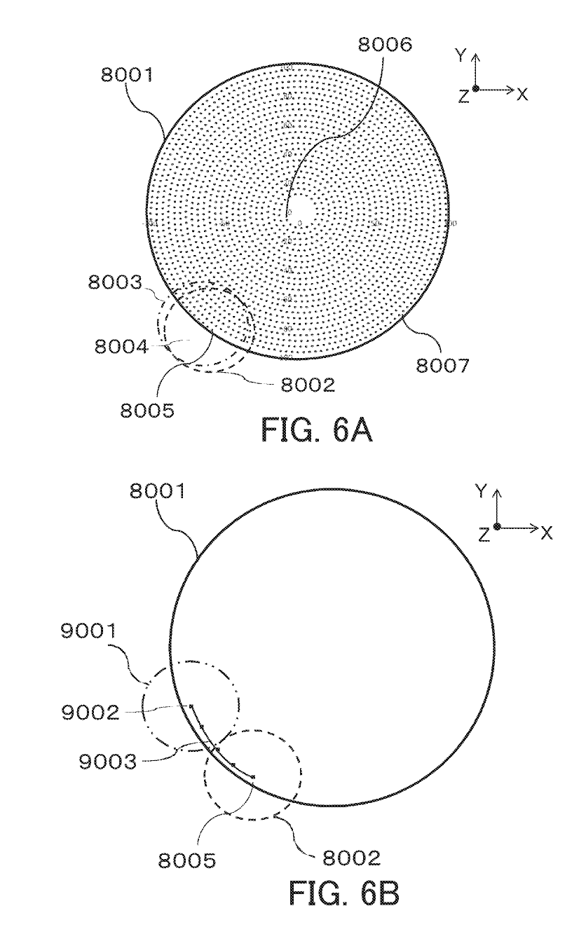

Calculation of the scanning speed in the case of a spiral scanning trajectory will be described. FIG. 6A illustrates a scanning trajectory along which the input imaging area (area of interest) is scanned. FIG. 6B illustrates two irradiation areas irradiated with the respective pulsed light beams and completely separated from each other.

The input imaging area is denoted by reference numeral 8001. In the present embodiment, the imaging area is circular. An area 8002 (enclosed by a dashed line) is irradiated with pulsed light. In the present embodiment, the irradiation area is shaped like a circle with a radius of 10 mm for simplification. An area 8003 (enclosed by a long dashed short dashed line) is irradiated with the next pulsed light beam. In an area 8004, the areas 8002 and 8003 corresponding to the consecutive pulsed light beams overlap. The scanning starts at a point 8005. The scanning ends at a point 8006. A dotted line 8007 represents a scanning trajectory corresponding to the scanning from the start point to the end point.

During the spiral scanning, an irradiation area 9001 depicted by a long dashed double-short dashed line in FIG. 6B does not overlap the irradiation area 8002. A central point of the irradiation area 9001 is denoted by reference numeral 9002. A scanning distance (hereinafter referred to as a distance A) from the start point 8005 to the central point 9002 is denoted by reference numeral 9003.

As seen in FIG. 2, when laser light has a wavelength of 756 nm and an irradiation frequency of 20 Hz, an identical area can be irradiated using a laser intensity equivalent to the laser intensity in the case of an irradiation frequency of 10 Hz, for 3.8 seconds since the start of irradiation. Therefore, for movement of the irradiation area, the irradiation area needs to move to the area 9001 before 3.8 seconds have elapsed since the irradiation of the irradiation area 8002. This allows a laser intensity corresponding to 10 Hz to be used even at a laser irradiation frequency of 20 Hz. In other words, the irradiation area needs to be moved to a position where the irradiation area does not overlap the irradiation area 8002 before 3.8 seconds have elapsed since the irradiation. Therefore, the control unit sets the scanning speed to a value meeting this condition.

In the present embodiment, the irradiation area is a circle with a radius of 10 mm. Since the laser light has a wavelength of 756 nm and an irradiation frequency of 20 Hz, light can be radiated at a laser intensity corresponding to 10 Hz for 3.8 seconds. Therefore, the scanning speed during the irradiation from the start point 8005 to the central point 9002 needs to be higher than A/3.8. In this case, when the scanning trajectory is spiral, the distance to the point where the irradiation areas do not overlap varies according to the position on the scanning trajectory irradiated with light. Therefore, the scanning speed is varied according to the scanning position in the spiral. In the present embodiment, the distance to the point where the irradiation areas do not overlap increases toward the center. Therefore, the scanning speed increases toward the center.

When the scanning speed is increased toward the center, the number of pulsed light irradiations may be reduced at the center. Thus, upon reaching a point 8006 (an end point for a case where the scanning proceeds from an outer circumference side to an inner circumference side), the scanning may be performed in the opposite direction. That is, a start point is set at the position 8006, and outward spiral scanning is performed toward the position 8005. The purpose in this case is to compensate for the insufficiency of signals acquired on the inner circumferential side (a central side of the spiral). Thus, the irradiation area need not necessarily reach the position 8005. Alternatively, the scanning in the opposite direction may be faster toward the outer circumferential side. Consequently, acquisition of data on areas near the center can be prioritized without the need to extend the time for which received signals are acquired.

The outward scanning starting at the point 8006 need not follow the trajectory of inward scanning from the point 8005 toward the point 8006, in the opposite direction. When clockwise inward scanning ends at the position 8006, counterclockwise scanning is performed toward the point 8006. Then, a strong inertia force acts on the supporting body 400. Particularly when the acoustic matching material contained in the supporting body 400 is a liquid, the surface of the matching material is disturbed, possibly hindering the photoacoustic measurement. Thus, the outward scanning is performed clockwise similarly to the inward scanning so as to allow the inward scanning and the outward scanning to be consecutively switched. This enables the disturbance of the liquid surface to be suppressed.

In this case, the irradiation area may be shaped like an ellipse, a rectangle, or the like in association with the optical system. Furthermore, laser light with a wavelength of 756 nm preferably has an intensity corresponding to 10 Hz. However, the effects of the present embodiment are produced so long as the intensity is higher than the smallest value of the MPE.

As described above, even with an increased irradiation frequency of laser light, determination of the scanning speed as in the case of Embodiment 2 allows radiation of the laser light at an intensity higher than the smallest value of the MPE with respect to the irradiation frequency. For example, laser light with a wavelength of 756 nm can be radiated using the highest MPE even when the irradiation frequency of the laser light is higher than 10 Hz. Thus, the SNR can be increased. Appropriate setting of the scanning speed and the scanning trajectory enables a preferential increase in the SNR of areas near the center of the spiral. As a result, the SNR increases to improve the contrast of resultant images, enhancing the capability of making diagnoses based on photoacoustic images.

### Embodiment 3

In the description of Embodiment 3, a certain segment is to be preferentially observed. A configuration in Embodiment 3 is similar to the configuration in Embodiment 1 and will thus not be described. For the description of a sequence of operations, focus is placed on differences from Embodiment 1.

<Operations of the Photoacoustic Apparatus>

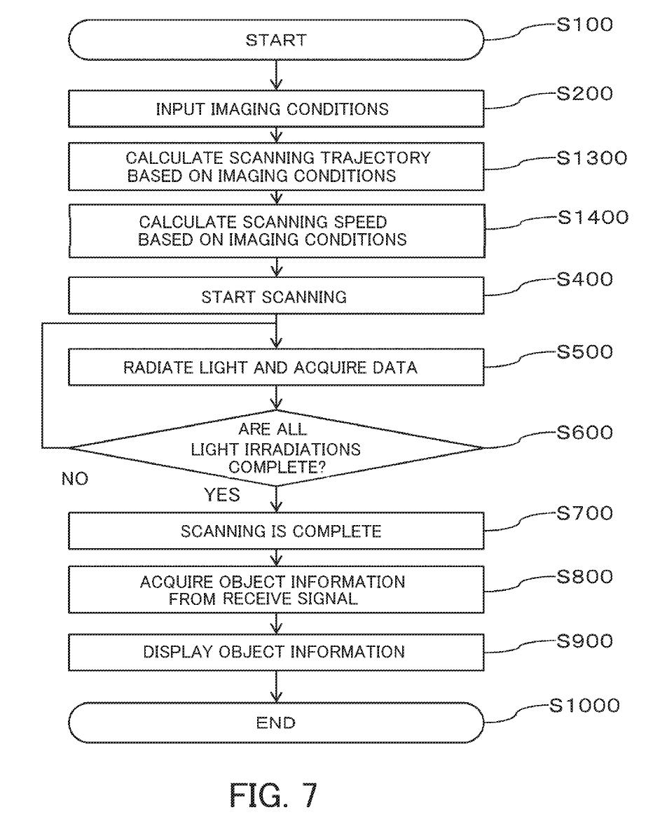

Now, using a flowchart in FIG. 7, operations of the embodiment of the present invention will be described with S200, S1300, and S1400 focused on.

(S200: Step of Inputting Imaging Conditions)

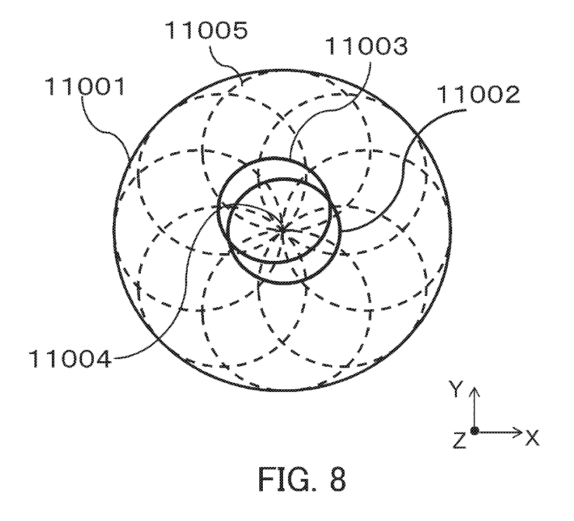

The user inputs imaging conditions. For one of the imaging conditions, a segment to be preferentially observed is input. By way of example, an image of the object E acquired by the shape acquiring unit 600 may be displayed on the display unit so that the user can input, to the apparatus, a segment in the image that is to be preferentially observed. The other imaging conditions are similar to the corresponding imaging conditions in Embodiment 1. The segment to be preferentially observed in this case is the center of an imaging area 11001 in FIG. 8.

(S1300: Step of Calculating a Scanning Trajectory based on the Imaging Conditions)

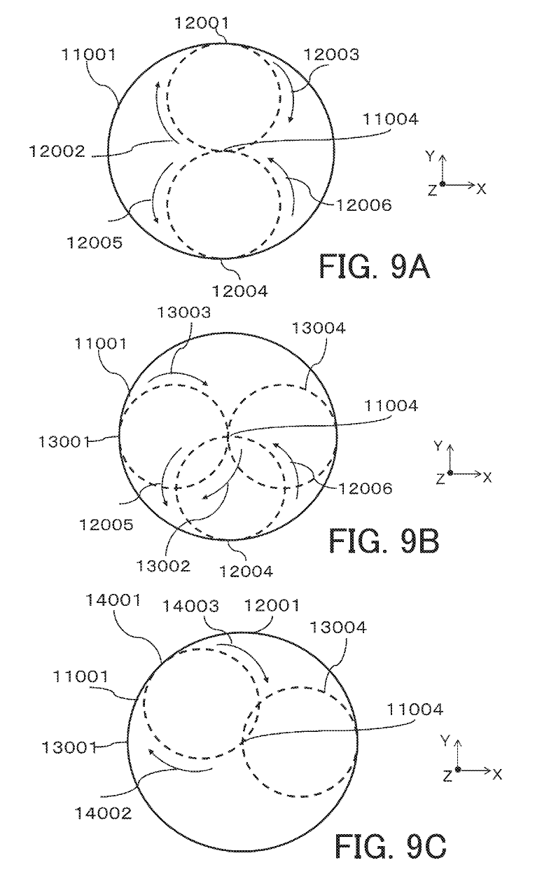

A scanning trajectory is calculated in a case where the segment to be preferentially observed is set. The present embodiment will be described using FIGS. 8 and 9. FIG. 8 illustrates a scanning trajectory obtained when the center of the imaging area is designated as a segment to be preferentially observed. FIGS. 9A to 9C are each an excerpt of a part of the scanning trajectory. The trajectory in this case refers to, for example, a line drawn by the center of a hemisphere during scanning.

In FIG. 8, an input imaging area is denoted by reference numeral 11001 and is circular in the present embodiment. An area 11002 is an irradiation area for a pulsed light beam and is shaped like a circle with a radius of 10 mm for simplification. An area 11003 is an irradiation area for the next pulsed light beam. The segment to be preferentially observed is denoted by reference numeral 11004. A calculated scanning trajectory is denoted by reference numeral 11005 and depicted by a dashed line.

In FIGS. 9A to 9C, the scanning trajectory intersects the imaging area at intersection points 12001 and 12004, 13001, and 14001. Scanning directions are denoted by reference numerals 12002, 12003, 12005, 12006, 13002, 13003, 14002, and 14003. An excerpt of a part of the scanning trajectory is denoted by reference numeral 13004.

Now, specific calculation of a scanning trajectory will be described. In this case, the scanning starts at the center 11004 of the imaging area 11001 depicted in FIG. 9A, and progresses in the direction 12002 toward the point 12001. Upon reaching the point 12001, the scanning progresses again in the direction 12003 toward 11004. In the present embodiment, the illustrated scanning trajectory is circular. However, the scanning trajectory may be shaped like an ellipse, a square, a rectangle, a polygon, or a straight line. Then, the scanning progresses in the direction 12005 from the point 11004 toward the point 12004 (that is, toward the farthest point on the circumference of the imaging area). Upon reaching the point 12004, the scanning progresses in the direction 12006 toward the point 11004.

As described above, in FIG. 9A, the scanning trajectory traces out a figure of eight including two circles. The two circles included in the figure of eight contact each other at the central point of the imaging area 11001 and are inscribed in the scanning area 11001. In this case, a segment with which the points 12001 and 12004 are connected together passes through the point 11004. This is hereinafter referred to as a figure-of-eight axis.

As seen in FIG. 9B, as soon as the trajectory reaches the point 11004, the scanning progresses in the direction 13002 toward the point 13001 located on the circumference of the imaging area at 90° left from the point 12004 with respect to the point 11004. Upon reaching the point 13001, the scanning progresses in the direction 13003 toward the point 11004. Similarly, the scanning is performed on the circumference 13004. Consequently, the scanning trajectory traces out a figure of eight with an axis located at an angle different from the angle at which the figure-of-eight axis is located in FIG. 9A.

As seen in FIG. 9C, the scanning progresses in the direction 14002 toward the point 14001 located on the circumference of the imaging area at an intermediate point between the points 13001 and 12001. Upon reaching the point 14001, the scanning progresses in the direction 14003 toward the point 11004. Consequently, the scanning trajectory traces out a figure of eight with an axis located at an angle different from both the angles at which the figure-of-eight axes are located in FIGS. 9A and 9B.

In this case, relative positional relations between the points 12001 and 13001 and 14001 are not limited to the relative positional relations illustrated in the present embodiment. The above-described operations are repeated to allow the scanning trajectory 11005 to be calculated. The scanning trajectory passes through the point 11004 positioned in the central portion of the area of interest a plurality of times. That is, whereas the spiral trajectory needs to increase the speed in the central portion, the scanning trajectory in FIGS. 9A, 9B, and 9C obviates the need to increase the speed because the connection point between the two small circles of the figure of eight aligns with the center of the area of interest. Trajectories of a plurality of figures of eight have a common intersection point, and the figures of eight are located at different angles, allowing a large area to be covered. As a result, the imaging area can be exhaustively scanned and the segment to be observed can be preferentially scanned. The case where the figures of eight have a common intersection point includes a case where intersection points lie in areas that are considered to be virtually identical (for example, if the object is the breast, within a circle with a diameter of 1 cm). The angle at which the axis is located is preferably inside the area of interest.

(S1400: Step of Calculating the Scanning Speed Based on the Imaging Conditions)

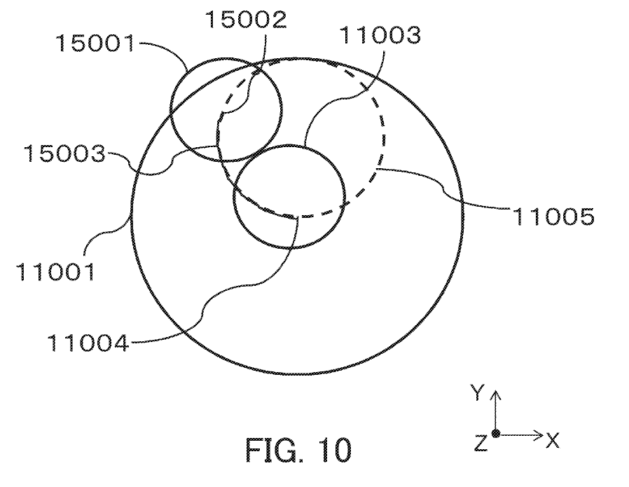

The scanning speed is calculated in a case where the scanning trajectory is calculated in S1300. FIG. 10 illustrates two irradiation areas irradiated with the respective pulsed light beams and completely separated from each other. That is, during the irradiation, an irradiation area 15001 does not overlap the irradiation area 11003. A central point of the irradiation area 15001 is denoted by reference numeral 15002. A scanning distance (hereinafter referred to as a distance B) from the start point 11004 to the central point 15002 is denoted by reference numeral 15003.

As seen in FIG. 2, when laser light has a wavelength of 756 nm and an irradiation frequency of 20 Hz, an identical area can be irradiated using a laser intensity equivalent to the laser intensity in the case of an irradiation frequency of 10 Hz, for 3.8 seconds since the start of irradiation. Therefore, the scanner 400 desirably moves to the irradiation area 15001, which does not overlap the irradiation area 11003, before 3.8 seconds have elapsed since the irradiation of the irradiation area 11003. Consequently, a laser light intensity equivalent to the intensity in the case of an irradiation frequency of 10 Hz can be utilized even at an irradiation frequency of 20 Hz.

In the present embodiment, the irradiation area is a circle with a radius of 10 mm, the laser light has a wavelength of 756 nm and an irradiation frequency of 20 Hz, and the laser light can be radiated at a laser intensity corresponding to 10 Hz for 3.8 seconds. Therefore, the scanning speed during the irradiation from the start point 11004 to the central point 15002 needs to be higher than B/3.8. The distance to the point where the irradiation areas do not overlap varies according to the size of the irradiation area, the arrangement of the irradiation start position and end position in the imaging area (area of interest), the shape of the trajectory (for example, a circular arc or a straight line), and the like. Therefore, the scanning speed needs to be varied according to the distance. The irradiation area may be shaped like an ellipse, a rectangle, or the like and may be set based on the optical system. The laser light preferably has an intensity corresponding to 10 Hz. However, the effects of application of the present embodiment can be enjoyed so long as the intensity is higher than the smallest value of the MPE determined by the wavelength and irradiation frequency of light radiated to the object.

Even with an increased irradiation frequency of laser light, determination of the scanning trajectory and the scanning speed as described above allows radiation of the laser light at an intensity higher than the smallest value of the MPE with respect to the irradiation frequency. Thus, the SNR can be increased. An increase in the SNR of the segment designated to be preferentially observed can be prioritized. As a result, the SNR increases to improve the contrast of resultant images, enhancing the capability of making diagnoses based on photoacoustic images. The principle of the present invention is applicable to various wavelengths and irradiation frequencies of light.

As described above, the present invention enables the SNR to be increased.

### Other Embodiments

The present invention may also be carried out by a computer (or a device such as a CPU or an MPU) in a system or an apparatus that implements the functions of the above-described embodiments by loading and executing a program recorded in a storage device. The present invention may also be carried out, for example, by a method including steps performed by a computer in a system or an apparatus that implements the functions of the above-described embodiments by loading and executing a program recorded in a storage device. The present invention may also be carried out by a circuit that implements one or more functions (for example, an ASIC). To accomplish this object, the program is provided to the computer, for example, through a network or various types recording media that could constitute the storage device (in other words, a computer readable recording medium that holds data in a non-transitory manner). Therefore, the scope of the present invention includes all of the computer (including a device such as a CPU or an MPU), the method, the program (including a program code and a program product), and the computer readable recording medium that holds the program in a non-transitory manner.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment (s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-088025, filed on Apr. 26, 2016, which is hereby incorporated by reference herein in its entirety.

What is claimed is:

1. An object information acquiring apparatus comprising:
an irradiating unit that irradiates an object with light with a first wavelength at a first irradiation frequency;
an element that receives an acoustic wave generated by the object irradiated with the light to output an electric signal;
a processing unit that acquires characteristic information on the object using the electric signal;
a scanning unit that changes a position of the irradiating unit relative to the object; and
a controlling unit that controls the scanning unit,
wherein the controlling unit controls the scanning unit such that an amount of light to which an identical area of the object is exposed is larger than a smallest value of a maximum permissible exposure at the first irradiation frequency.

2. The object information acquiring apparatus according to claim 1, wherein the controlling unit controls the scanning unit such that a period during which the identical area of the object is irradiated with the light is shorter than a first period, and
the first period is a period in which the maximum permissible exposure at the first irradiation frequency is higher than the smallest value of the maximum permissible exposure at the first irradiation frequency.

3. The object information acquiring apparatus according to claim 2, wherein the first wavelength is 756 nm,
the first irradiation frequency is 20 Hz, and
the first period is a time that is not more than 10 seconds.

4. The object information acquiring apparatus according to claim 2, wherein the first period is a time that is not more than 3.8 seconds.

5. The object information acquiring apparatus according to claim 1, wherein the controlling unit controls the scanning unit such that irradiation areas irradiated with the light during two consecutive irradiations do not overlap.

6. The object information acquiring apparatus according to claim 1, further comprising a supporting body configured to support the irradiating unit and a plurality of the elements,
wherein the controlling unit allows the scanning unit to vary the position of the supporting body relative to the object.

7. The object information acquiring apparatus according to claim 6, wherein the controlling unit controls the scanning unit such that the scanning unit moves the supporting body along a spiral scanning trajectory.

8. The object information acquiring apparatus according to claim 6, wherein the controlling unit controls the scanning unit such that the scanning unit moves the supporting body along a scanning trajectory of a plurality of figures of eight that has axes located at different angles and that has a common intersection point.

9. The object information acquiring apparatus according to claim 8, further comprising an input unit configured to accept an input from a user relating to an area of interest,
wherein the controlling unit controls the scanning unit such that the common intersection point is located inside the area of interest.

10. An object information acquiring method comprising:
an irradiating step of irradiating, by an irradiating unit, an object with light with a first wavelength at a first irradiation frequency;
a receiving step of receiving, by an element, an acoustic wave generated by the object irradiated with the light to output an electric signal;
a processing step of acquiring characteristic information on the object using the electric signal;
a scanning step of varying, by a scanning unit, a position of the irradiating unit relative to the object; and
a controlling step of controlling the scanning unit,
wherein the controlling step controls the scanning unit such that an amount of light to which an identical area of the object is exposed is larger than a smallest value of a maximum permissible exposure at the first irradiation frequency.

11. The object information acquiring method according to claim 10, wherein the controlling step controls the scanning unit such that a period during which the identical area of the object is irradiated with the light is shorter than a first period, and
the first period is a period in which the maximum permissible exposure at the first irradiation frequency is higher than the smallest value of the maximum permissible exposure at the first irradiation frequency.

12. The object information acquiring method according to claim 11, wherein the first wavelength is 756 nm,
the first irradiation frequency is 20 Hz, and
the first period is a time that is not more than 10 seconds.

13. The object information acquiring method according to claim 11, wherein the first period is a time that is not more than 3.8 seconds.

14. The object information acquiring method according to claim 10, wherein the controlling step controls the scanning unit such that irradiation areas irradiated with the light during two consecutive irradiations do not mutually overlap.

15. The object information acquiring method according to claim 10, wherein the controlling step allows the scanning unit to vary the position of a supporting body relative to the object, and
the supporting body is configured to support the irradiating unit and a plurality of the elements.

16. The object information acquiring method according to claim 15, wherein the controlling step controls the scanning unit such that the scanning unit moves the supporting body along a spiral scanning trajectory.

17. The object information acquiring method according to claim 15, wherein the controlling step controls the scanning unit such that the scanning unit moves the supporting body along a scanning trajectory of a plurality of figures of eight that has axes located at different angles and that has a common intersection point.

18. The object information acquiring method according to claim 17, further comprising an inputting step of accepting, by an input unit, an input from a user relating to an area of interest,
wherein the controlling step controls the scanning unit such that the common intersection point is located inside the area of interest.
